(19) United States
(12) Patent Application Publication
(10) Pub. No.: US 2009/0105684 A1
(43) Pub. Date: Apr. 23, 2009

(54) MEDICATION PORT FOR MEDICAL FLUID CONTAINER

(75) Inventors: Patrick Balteau, Evelette (BE); Giampiero Pesci, Tirano Sondrio (IT); Jean Luc Dewez, Bousval (BE)

Correspondence Address: BAXTER HEALTHCARE CORPORATION, 1 BAXTER PARKWAY, DF2-2E, DEERFIELD, IL, 60015, US

(73) Assignees: BAXTER INTERNATIONAL INC., Deerfield, IL (US); BAXTER HEALTHCARE S.A., Zurich (CH)

(21) Appl. No.: 11/877,517

(22) Filed: Oct. 23, 2007

(51) Int. Cl. A61B 19/00

(52) U.S. Cl. 604/415

(57) ABSTRACT

A medication port for a flexible container is disclosed. The port is made from one or from two materials. If made from two materials, the parts are integrally joined and cannot be separated without destroying the port. The port is sterilizable by steam or gamma-irradiation, or preferably both. The port is attached to the container by a technique for integral joining, such as molding or ultrasonic welding. The port may also be equipped with a removable, peelable film attached to the housing.

### BACKGROUND

The present disclosure generally relates to a medication port, an apparatus and a method for using a medication port for injecting or withdrawing a liquid from a container. More specifically, the present disclosure relates to improved materials and material designs for the medication port. Additionally, the present disclosure provides an apparatus and a method that may be used for the parenteral administration of a medical solution while providing for injection of an additional component, for example a drug, into the solution.

It is generally known that an individual may require a form of medication. Often, the medication must be administered to the patient parenterally, for example intravenously. For example, it may be impractical or impossible to administer medication orally to the patient, for example when the patient is unconscious or when a large volume of medication is to be delivered. Further, the patient may require prolonged, constant and/or immediate medication that may only be administered parenterally. Of course, numerous other reasons exist for parenterally medicating a patient.

Medical infusion solutions are typically stored in containers constructed from, for example, flexible plastic or glass. An administration port on the container is adapted to connect to an administration set (i.e., IV tubing) that is connected to a patient's vein. The mixed solution then flows from the administration port, through the IV, and into the patient's bloodstream. Other parenteral administration routes may also be used to deliver medication or other therapeutic fluid treatment to a patient. For example, medications and hydration fluids may be administered subcutaneously. As a further example, patients suffering from end stage renal disease may receive a fluid based therapy such as peritoneal dialysis.

Further, it is generally known to provide a medication port on the medical solution container through which drugs and/or other solutions may be administered. The medication port typically includes a resilient septum or membrane that may be pierced by a needle or cannula to provide sterile transfer of fluid into or out of the container. For example, hospital patients are often given an IV solution such as dextrose or saline to ensure that an administration route is already available if medication is required. Such medications are frequently delivered by injecting them into an access port on the IV solution container. As a further example, a diabetic patient receiving an infusion of a glucose-based peritoneal dialysis solution may need to add insulin to the solution to avoid a dangerous increase in blood sugar. Additionally, dialysis patients occasionally need to add other medications such as heparin or antibiotics to their dialysis solution to address acute conditions that have developed during their therapy.

Known medication ports are often constructed as one-way valves which allow the addition of a medication to a container. However, known medication ports may be difficult to maintain in a sterile condition once used. Bacteria, viruses, dirt, and other potentially harmful substances may be present on the surface of the septum, membrane or container. As a result, such substances may be inadvertently introduced into the solution.

Typically, a medication port is constructed or attached to a container either as an up-port or as a side-port. The up-port is generally located at a distal end of the container while the side port is located on a sidewall of the container. It is also generally known to provide a septum, also referred to as a bung, within an opening or port of the container. The septum, which is typically constructed of a resilient material such as an elastomer, prevents liquid inside the container from leaving the container. Additionally, the septum reduces the risk of foreign substances from entering the container. Further, known septa often may be pierced by a needle, cannula, tube or other object to establish fluid communication with the liquid in the container. Insertion of the fluid conduit may be facilitated by providing a pre-cut slit in the septum, which may extend all or part of the way through the thickness of the septum. Preferably the septum can be repeatedly pierced without compromising the integrity or sterility of the container.

A cap is often incorporated with the medication port to enclose and protect the septum. However, caps often completely surround the entire opening to the container. As a result, known caps are often bulky, expensive and inefficient. For instance, larger caps require more material to produce and add weight and/or complexity to the entire apparatus. In addition, personnel handling a cap may contaminate the cap while removing it. A need therefore exists for a medication port as well as an apparatus and a method for injecting or withdrawing a liquid from a container to overcome deficiencies of known ports and apparatus and methods using such a port. Additionally, a need exists for a medication port that allows a liquid to be introduced to a container in a sterile environment.

U.S. Pat. No. 6,994,699, assigned to the assignees of the present application, discloses a medication port assembly that includes a housing with a removable cover, a septum mounted within the housing, and a locking ring holding the septum in place. It is desirable to provide a medication port that performs comparably to this medication port while simplifying assembly. The present disclosure provides medication ports satisfying this need.

### SUMMARY

A first embodiment disclosed herein is a medication port for a medical container. The port includes a housing having a peripheral wall defining an interior, said housing made from a medical grade plastic or elastomeric material, the housing further including a surface for sealing against the container, and a septum made from a medical grade plastic or elastomeric material, the septum integrally attached to the housing, wherein the port is suitable for sterilizing by known wet and/or dry sterilization methods.

Another embodiment includes a medication port for a medical container. The port includes a housing made from a medical grade plastic or elastomeric material, the housing further including a surface for sealing against the container, and a septum made from the same material and integrally attached to the housing.

Another embodiment is a medical fluid container assembly. The medical fluid container includes a flexible film sheet having at least an inner film layer and an outer film layer, the film sheet formed into a medical fluid container sealed or folded closed on four edges. The medical fluid container assembly also includes an administration port attached near one end of the medical fluid container, and a medication port attached to one side of the medical fluid container, the medication port including a housing made from a medical grade plastic or elastomeric material, the housing further including a surface for sealing against the medical fluid container and a septum made from a medical grade plastic or elastomeric material, the septum integrally attached to the housing, wherein the medication port is suitable for sterilization by one or more known sterilization methods, by at least steam and gamma-irradtion methods.

Another embodiment includes a method of making a medication port for a medical container. The method includes steps of forming a housing from a medical grade plastic or elastomeric material, forming a septum from a medical grade plastic or elastomeric material, and integrally joining the housing and septum wherein at least the housing and the septum are suitable for sterilizing by at least one of steam and gamma-irradiation methods.

Additional features and advantages are described herein, and will be apparent from, the following Detailed Description and the figures.

### BRIEF DESCRIPTION OF THE FIGURES

FIG. 1 is a perspective view of a medical container with a port embodiment according to the present invention;

FIG. 2 is a perspective view of a first embodiment of a port as described herein;

FIG. 3 is a cross-sectional view of the port of FIG. 2;

FIG. 4 is a perspective view of a second embodiment of a port;

FIG. 5 is a cross-sectional view of the port of FIG. 4;

FIG. 6 is a cross-sectional view of a third embodiment of a port; and

FIG. 7 is a flowchart for a method of manufacturing a medication port.

### DETAILED DESCRIPTION

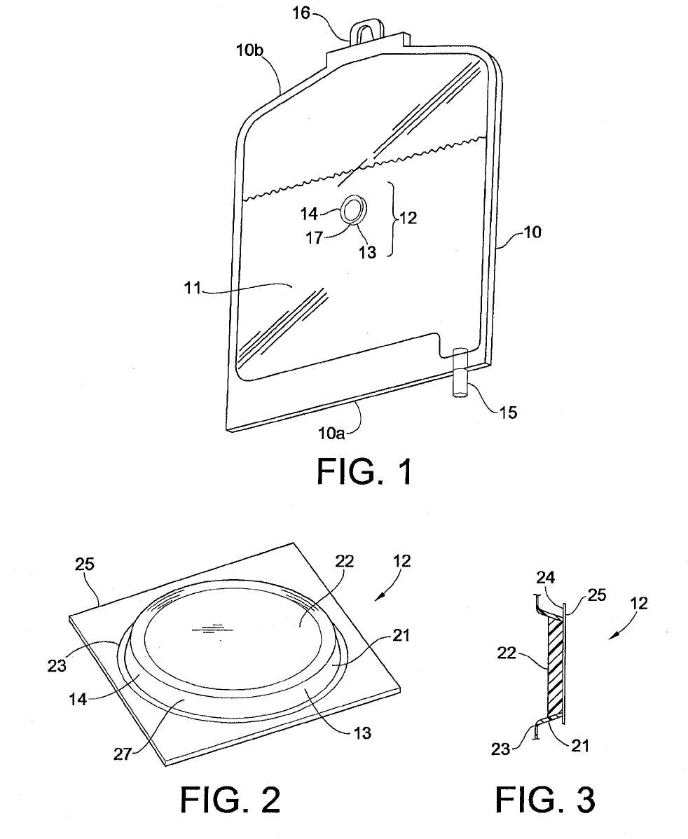

The present invention generally relates to a port, a container and a method for accessing a container for injecting or withdrawing a liquid from the container. Additionally, the present invention relates to a container having a port. More specifically, the present invention relates to a port, a container and a method for accessing a container to introduce a drug into the port. Referring now to the drawings, FIG. 1 illustrates a perspective view of a container 10 having a first end 10a and a second end 10b. The container 10 may be peripherally sealed and may have a medical solution 11 or other liquid in an interior of the container 10. Container 10 may be constructed of a flexible material, such as a PVC or non-PVC material, sealed on all four sides to constitute a sturdy, leak-proof container. Such containers are generally known and, as such, will not be described in further detail herein.

Container 10 has a medication port 12 having an inner portion 13 adjacent the container at interface 17 and an outer portion 14, the outer portion being the portion of the port that is most distal from the inner portion 13. Medication port 12 of the present invention may be a side port as shown or could also be an up-port, located at a distal end of container 10. Container 10 is illustrated in a position as is common in actual use, positioned up-right with medication port 12 elevated above an administration port 15 in actual use. Additionally, container 10 may also include a hanger 16 for use with a hook to hang the container in an elevated position at or near a patient. When the container 10 is positioned at or near the patient, gravity may force the liquid 11 inside the container through the administration port 15 to the patient. Preferably, the administration port 15 may be located remotely from the medication port 12 as illustrated in the embodiment shown in FIG. 1.

The medication port is affixed to the container as shown in FIG. 1. Assembly may be accomplished by any of several recognized techniques, such as ultrasonic welding, heat sealing or plastically welding to the container. Any of these methods should result in an integral attachment of the port to the container, that is, the port cannot be removed from the container without destroying either the container or the port, or both.

A first embodiment of a port is depicted in FIGS. 2-3. Port 12 includes a housing 21, a septum 22, and a lip 23 for sealing against the container to which the port is attached. The top surfaces of the septum and the outer portion are preferably aligned, i.e., they lie in about the same plane. The walls 27 of the housing may be parallel, or they may be as shown, slightly tapered inwardly, so that the circumference at the bottom portion 13 is slightly larger than the circumference at the top portion 14. As mentioned above, inner portion 13 is the portion of the medication port that is adjacent the container to which the port is attached. The housing and septum are preferably made of materials that are easily sterilized, so that use of the port does not introduce foreign matter or undesirable microorganisms into the port or the container. To protect the septum from contaminants, the port may also include an outside seal 25. Outside seal 25 is preferably adhered to the outer portion 14 of the port. The seal is thus preferably peelable, i.e., a peelable outer seal layer or peelable film. This seal helps to maintain the sterility of the port. The seal is typically attached by melting a layer of polymer film onto the portions of the housing surrounding the septum. Suitable peelable films include a peel seal layer containing an alloy of one or more polyolefins with a thermoplastic elastomer, such as an 80% polypropylene/20% SEBS alloy. For example, one suitable film is the peelable film described in U.S. Pat. No. 6,319,243, which consists of layers of polyester, maleated EVA, EVA and polypropylene/SEBS. Other suitable materials include the multilayer films described in European Patent No. EP 1 139 899 B1, assigned to the assignee of the present application. One such film includes a polypropylene skin layer, a nylon core layer, and a peelable seal layer containing a propylene-ethylene random copolymer, linear low density polyethylene, and SEBS block copolymer. Another suitable film disclosed in the same patent includes a seal layer containing SEBS and two polypropylenes with different melting temperatures. Each of the aforementioned patents is incorporated herein by reference to the extent not inconsistent with the rest of this disclosure.

In the embodiment of FIGS. 2-3, the housing 21 and the septum 22 are preferably integrally attached. The septum is not captured between lips of the housing. Instead, the septum is retained by virtue of its integrity with the housing. The integrity is a result of the method of manufacture, which may occur in several ways. The septum may be made separately and inserted into a tool, such as an injection-molding tool or rotational-molding tool. The housing is then molded around the septum. Alternatively, the housing may be molded first using an annular die, after which the central portion of the die may be removed so that the septum may be formed directly within the housing by injection molding. The housing and septum may also be made together as a single part, i.e., they are molded from a single material in a single process.

In some embodiments, two materials may be used, one for the septum and one for the housing. In other embodiments, a single material may be used for both the housing and the septum. In embodiments using two materials, the housing is preferably made from a medical grade plastic that is suitable for sterilization, such as polypropylene. The polypropylene may also be blended with polymers such as ultra low density polyethylene, linear low density polyethylene, or thermoplastic elastomers such as SEBS (styrene-ethylene/butene-styrene block copolymer) or SEPS (styrene-ethylene/propylene-styrene block copolymer). Suitable thermoplastic elastomers include the Kraton® G series from Kraton Polymers and the Cawiton Med series available from Wittenburg B.V. Other plastic and elastomeric materials suitable for the housing include polypropylene modified with EVA (such as Escorene®); EPDM (such as Santoprene™ TPV); or silicone rubber (such as TPSiV™). Usually, about 15-30% modifier is sufficient. Many other medically acceptable materials may also be used. In an embodiment, the housing is made of a blend of approximately 55-60% polypropylene impact copolymer, 10-20% EVA and 15-25% SEBS; for example, the housing may have the following composition (identified in the data below as PL18016):

| | |
|---|---|
| BP Solvay BP401-CA20 polypropylene | 59% |
| Cawiton Med 712/1 SEBS | 23% |
| ExxonMobil ESCORENE ® FL 00328 EVA | 15% |
| Medical grade polyethylene colorant | 3% |

The inclusion of a colorant in the housing composition creates a strong visual contrast between the housing and the septum, which helps certain visually impaired patients to locate and use the medication port.

The septum is typically made from a softer, elastomeric material. Examples of materials suitable for the septum include thermoplastic elastomers such as SEBS (styrene-ethylene/butene-styrene) or SEPS (styrene-ethylene/propylene-styrene), examples of which include the Kratone® G series. Other suitable elastomeric materials include polypropylene/EPDM blends, such as Santoprene™ TPV; silicone rubber, such as TPSiV™; and alpha-olefin elastomers, such as VISTAMAXX™. VISTAMAXX™ is ExxonMobil's trademark for a family of polyethylene and polypropylene elastomers with a degree of crystallinity. Any of these polymers may be used alone or may be blended with polyisoprene (PI), styrene-isoprene-styrene block copolymer (SIS), or other polymers. Typically, the PI or SIS materials are about 0-30% of the total polymer. This softness or flexibility helps the septum to reseal after it is punctured by a needle to inject the medication into the container. In an embodiment, the septum is a food/medical grade thermoplastic elastomer such as THERMOLAST® K TF3STE available from Kraiburg TPE, or MARFRAN® M1/55, a SEBS thermoplastic elastomer available from VTC Franceschetti Elastomeri, Corte Franca, Italy.

Where the septum and housing are made of different materials, it is desirable that both parts contain at least one common or similar material to facilitate adhesion of the septum to the housing. For example, both components may contain a material that begins to melt close to the injection molding temperature or the sterilization temperature. This allows an adhesive bond to form between the components. In an embodiment, both the housing and the septum contain a styrene-hydrocarbon block thermoplastic elastomer. Other bonding methods may also be used, such as by ultrasonic welding, i.e., holding the two parts adjacent each other and vibrating them very rapidly with a horn that transmits the ultrasonic energy. The two parts may also be joined integrally by plastic welding, that is, a process in which a narrow bead of material of one of the parts, or a bead of a third material, is melted to form a “weld” between the parts. The term integral is thus used in the sense that the septum and the housing may only be separated from each other by destroying the assembled port.

Experimental results for a number of medication ports are tabulated in Table 1 below.

| | | Leak rate | | |
|---|---|---|---|---|
| Housing | | after puncturing at 8 psi | | |
| Material | Septum Material | 1 time | 5 times | 10 times |
| PL18016 | Kraiburg TF3STE | 0/15 | 0/15 | 2/15 |
| PL18016 | Marfran M1 55 | 7/10 | 7/10 | 9/10 |
| Kraiburg | Kraiburg TF3STE | 1/10 | 3/10 | 3/10 |
| TF3STE | | | | |
| Marfran M1 55 | Marfran M1 55 | 0/10 | 6/10 | 7/10 |

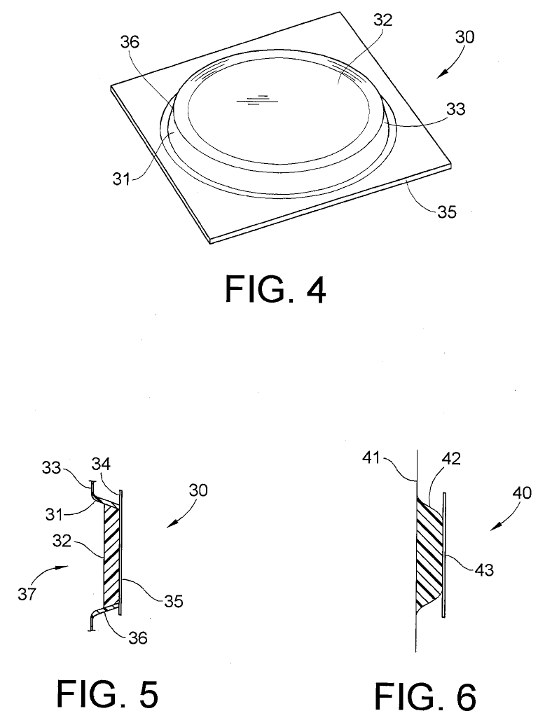

Another embodiment of a port is depicted in FIGS. 4-5. Port 30 includes a housing 31, a septum 32 and a sealing lip 33. The port may also include a peelable film 35, which is held onto the port. The peelable film is preferably tamper-evident and easily removed so that a medication can be injected through the septum and into the container. Port 30 has a lower profile, i.e., the sidewalls 36 have less height than the sidewalls of port 12. In one embodiment, the walls 36 are only slightly higher than the thickness of septum 32, so that the height of the gap 37 between the under side of lip 33 (the level of the container) and the bottom of the septum 32 is about 1.6 mm (about 1/16 of an inch).

In another embodiment, depicted in FIG. 6, a flat port 40 has virtually no gap between the bottom of septum 42 and the underside of lip 41. Port 40 also includes a peelable film layer 43.

The housing and septum embodiments of FIGS. 4-6 may be made by the methods described above, and may also be made from a single material and in a single processing step, such as a plastics molding operation. A needle, of course, must be able to penetrate the septum to deliver a medication to the container. Thus, when made from a single material, the housing and septum, i.e. the major components of the port, are preferably elastomeric. Alternatively the port could be made from a relatively high density closed cell foam.

The materials preferred for the single-material embodiments include thermoplastic elastomers based on styrene block copolymers with polybutadiene, polyisoprene, and poly-isoprene/butadiene. Examples include THERMOLAST® K TF3STE and TF4STA thermoplastic elastomers available from Kraiburg TPE. The inventors have found that single-material medication ports work well when the material has a Shore A hardness from about 35 to 65, preferably about 35 to 50, and most preferably about 45 Shore A. Materials with this hardness, or rather softness, are easily flexed and have no trouble admitting a needle to deliver a medication. These materials typically also have sufficient strength to resist normal handling and use. Typical tensile strengths range from about 1.6 ksi (6.5 N/mm²) to about 2.7 ksi (11.0 N/mm²), preferably about 1.6 ksi (6.5 N/mm²) to about 2.45 ksi (10 N/mm²), and most preferably about 2.2 ksi (9.0 N/mm²). Other materials may be used and elastomers or plastics with other strengths may also be used.

Following the assembly of the port onto the container as described above, the container may be filled with a medical solution and terminally sterilized by methods known in the art, such as dry or moist heat sterilization.

Thus, the medication port provides a convenient means for delivering an additional medication, such as heparin, insulin, an anesthetic, an antibiotic, and so forth into the medical solution without compromising the sterility of the solution in the container. The port also allows medical personnel to withdraw a sample of the liquid in the container, before or after a medication is added to the container.

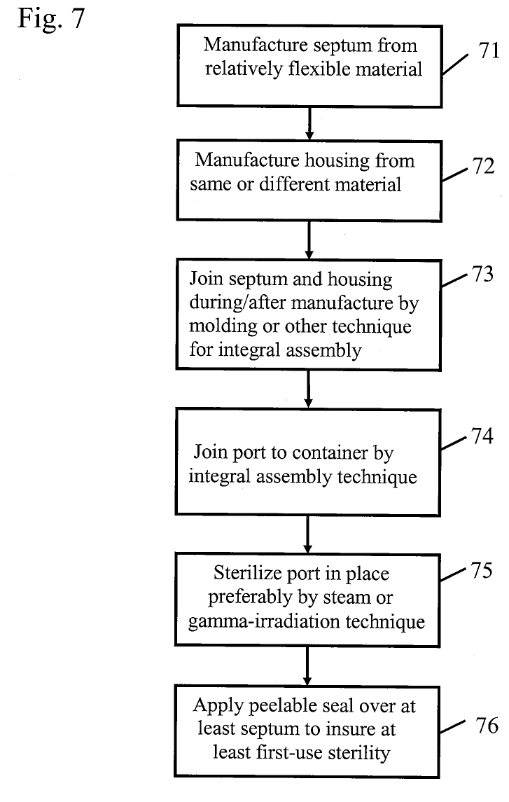

A flowchart for a method of manufacturing a medication port according to the above teachings is presented in FIG. 7. In one method, a septum for a medication port is manufactured 71 from a relatively flexible material, as discussed above. A housing for the medication port is manufactured 72 from the same or from different materials. As also described above, the septum and the housing are integrally joined 73, by manufacturing them together in the above steps or by manufacturing them separately and then joining them by adhesion, sonic welding, plastic welding, or any other useful technique. The port is then affixed 74 to the container of medical fluid by a technique that results in an integral bond between the container and the port. A peelable film may then be applied 76 over the port, at least over the septum, to ensure that the septum remains sterile at least for the first use of the port for adding a medication to the container through the port. The port is then sterilized 75 in place, preferably by steam or irradiation technique, to insure the sterility of the port after it has been joined to the container.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A medication port for a medical container, the port comprising:
a housing having a peripheral wall defining an interior, said housing made from a blend of approximately 55-60% polypropylene impact copolymer, 10-20% ethylene vinyl acetate and 15-25% styrene-ethylene-butene-styrene block copolymer, the housing further comprising a surface for sealing against the container; and
a septum made from a medical grade plastic or elastomeric material, the septum integrally attached to the housing, wherein the port is sterilizable by steam or gamma-irradiation methods.

2. The port of claim 1, wherein the septum is cohesively bonded to the housing by a technique selected from the group consisting of rotational molding, insert molding, sonic welding, plastic welding, and adhering with a medically acceptable permanent adhesive.

3. The port of claim 1, wherein the septum comprises a thermoplastic elastomer.

4. (canceled)

5. The port of claim 1, wherein the septum comprises a polypropylene-based thermoplastic elastomer selected from alloys of polypropylene with EPDM rubber, silicone rubber, or an alpha-olefin elastomer.

6. The port of claim 5, wherein the septum further comprises 0-30% of an elastomer selected from polyisoprene and block copolymers of styrene with butadiene, isoprene or a mixture thereof.

7. The port of claim 1, wherein the housing and septum are made from one material having a Shore A hardness from about 35 to about 65.

8. The port of claim 7, wherein the material is selected from the group consisting of thermoplastic elastomers based on styrene block copolymers with butadiene, isoprene, and mixtures thereof.

9. The port of claim 7, wherein the single material is a composite single material made from at least two thermoplastic elastomers.

10. The port of claim 1, further comprising a peelable film adhered to the housing and covering the septum.

11. The port of claim 10, wherein the peelable film is not in contact with the septum.

12. The port of claim 10, wherein the peelable film comprises at least one external layer comprising about 10% to about 40% by weight of a thermoplastic elastomer and about 60% to about 90% by weight of one or more polyolefins.

13. A medication port for a medical container, the port comprising:
a housing made from a material selected from the group consisting of thermoplastic elastomers based on styrene block copolymers with polybutadiene, polyisoprene, and poly(isoprene-co-butadiene) wherein the material has a Shore A hardness from about 35 to about 65, the housing further comprising a surface for sealing against the container; and
a septum made from the same material and integrally formed with the housing.

14. (canceled)

15. The port of claim 13, further comprising a peelable film attached to the housing.

16. The port of claim 15, wherein the peelable film comprises at least one external layer comprising about 10% to about 40% by weight of a thermoplastic elastomer and about 60% to about 90% by weight of one or more polyolefins.

17. The port of claim 15, wherein the peelable film is tamper- or use-evident.

18. A medical fluid container assembly, comprising,
a flexible film sheet having at least an inner film layer and an outer film layer, the film sheet formed into a medical fluid container sealed or closed on four edges;
an administration port attached near one end of the medical fluid container; and
a medication port attached to one side of the medical fluid container, the medication port comprising a housing made from a medical grade plastic or elastomeric material, the housing further comprising a surface for sealing against the medical fluid container and a septum made from a medical grade plastic or elastomeric material, the septum integrally attached to the housing, wherein the medication port is suitable for sterilizing by at least steam and gamma-irradiation methods.

19. A method of making a medication port for a medical container, the method comprising:
forming a housing from a medical grade plastic or elastomeric material;
forming a septum from a medical grade plastic or elastomeric material; and
integrally joining the housing and septum, wherein at least the housing and the septum are suitable for sterilizing by at least one of steam and gamma-irradiation methods.

20. The method of claim 19, wherein the housing and septum are formed together in a single process and are made from a single material.

21. The method of claim 19, wherein the housing and septum are formed together in a single process and made from a single material, the material having a Shore A hardness from about 35 to about 65.

22. The method of claim 19, wherein the housing and septum are formed in separate processes and further comprising a step of integrally attaching the septum to the housing, the step of integrally attaching comprising rotational molding, insert molding, sonic welding, plastic welding, and adhering with a medically-appropriate adhesive.

23. The method of claim 19, further comprising adhering a peelable film to the septum, wherein the peelable film is optionally tamper-evident.

24. The method of claim 19, further comprising joining the port to a medical fluid container.
